(19) United States
(12) Patent Application Publication
(10) Pub. No.: US 2011/0270084 A1
(43) Pub. Date: Nov. 3, 2011

(54) SURGICAL NAVIGATION APPARATUS AND METHOD FOR SAME

(76) Inventors: Seung Wook Choi, Gyeonggi-do (KR); Min Kyu Lee, Gyeonggi-do (KR)

(21) Appl. No.: 13/144,225

(22) PCT Filed: Feb. 8, 2010
(86) PCT No.: PCT/KR10/00764

§ 371 Date: Jul. 12, 2011

(30) Foreign Application Priority Data
Feb. 12, 2009 (KR) 10-2009-0011256
Feb. 25, 2009 (KR) 10-2009-0015652

(51) Int. Cl. A61B 6/00

(52) U.S. Cl. 600/427

(57) ABSTRACT

A surgical navigation apparatus and a method of operating the surgical navigation apparatus are disclosed. The surgical navigation apparatus includes: a first aligning unit configured to align a position of a patient with reference image data by using patient position data and the reference image data of the patient generated by image-taking before surgery; a second aligning unit configured to align the patient position data and comparative image data in real time, where the comparative image data is received from an image-taking unit; and an image processing unit configured to align the comparative image data and the reference image data in real time by using the patient position data. The surgical navigation apparatus can provide in real time images of the lesion taken during surgery, so that these images may be compared with those taken before surgery, to enable more accurate surgery and also provide greater convenience for the surgeon.

### CROSS-REFERENCE TO RELATED APPLICATIONS

This application is the National Phase of PCT/KR2010/000764 filed on Feb. 8, 2010, which claims priority under 35 U.S.C. 119(a) to Patent Application No. 10-2009-0011256 filed in the Republic of Korea on Feb. 12, 2009, and Patent Application No. 10-2009-0015652 filed in the Republic of Korea on Feb. 25, 2009, all of which are hereby expressly incorporated by reference into the present application.

### BACKGROUND

The present invention relates to a medical device and method, more particularly to a surgical navigation apparatus and a method of operating the surgical navigation apparatus.

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open so that an internal organ, etc., may be treated, reconstructed, or excised, can entail problems of blood loss, side effects, pain, scars, etc., and thus current methods of surgery that involve the use of surgical robots are currently regarded as popular alternatives.

Among conventional methods of surgery, image-guided surgery (IGS) is a method of tracking the position of a surgical tool within the operating room and visualizing it superposed over a diagnosis image, such as a CT and an MR image, etc., of the patient, to thereby increase the accuracy and safety of the surgery. FIG. 1 illustrates a surgical navigation apparatus according to the related art. Using an infrared camera 101, the surgical navigation system 100 identifies the position of an infrared reflector 103 that is attached to a probe 102, and the patient's lesion seen from the position of the probe 102 is shown on the display unit 104 of the surgical navigation system 100 in a corresponding portion on a 3-dimensional image data pre-stored in the surgical navigation system 100. To observe the patient's lesion with greater detail, a surgical microscope 105 can be used.

However, in a surgical navigation apparatus according to the related art, not all of the instruments actually used in surgery have position probes mounted thereon, and therefore a certain probe capable of position detection has to be used to achieve position detection. Also, the surgical navigation apparatus may be used frequently during position detection in the early stages of surgery, but when the position detection is completed and the actual surgery has commenced, the pre-stored image data may be different from or may be altered from the image data of the actual surgical site, and thus the surgical navigation apparatus may not be used as often.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a surgical navigation apparatus and its operating method by which an image of the lesion taken during surgery can be provided in real time and compared with an image taken before surgery. Another aspect of the present invention is to provide a surgical navigation apparatus and its operating method by which the current position of an endoscope and the 3D forms of the surrounding structures can be provided in juxtaposition with an image taken before surgery, to thereby enable more accurate surgery and also provide greater convenience for the surgeon.

One aspect of the present invention provides a surgical navigation apparatus that includes: a first aligning unit configured to align a position of a patient with reference image data by using patient position data and the reference image data of the patient generated by image-taking before surgery; a second aligning unit configured to align the patient position data and comparative image data in real time, where the comparative image data is received from an image-taking unit; and an image processing unit configured to align the comparative image data and the reference image data in real time by using the patient position data.

The image processing unit can align the comparative image data and the reference image data by using the patient position data and robot position data of a robot arm coupled with the image-taking unit.

Also, the image processing unit can control a display unit to output the reference image data and the comparative image data aligned with the patient position data.

Also, the image processing unit can align the comparative image data and the reference image data by using a distance from the robot arm, an extending direction, and a viewing direction of the image-taking unit.

Here, the image-taking unit can generate distance information for an object of image-taking by using a multiple number of lenses which each has a different parallax or by using one lens and taking images of the object while moving.

Another aspect of the present invention provides a method of operating a surgical navigation apparatus, by which the surgical navigation apparatus processes an image in real time during surgery. The method includes: aligning a position of a patient with reference image data by using patient position data and the reference image data of the patient generated by image-taking before surgery; aligning the patient position data and comparative image data in real time, where the comparative image data is received from an image-taking unit; and aligning the comparative image data and the reference image data in real time by using the patient position data.

Here, the reference image data can include data regarding a diagnosis image of the patient generated by image-taking before surgery, and the reference image data and the comparative image data can be 2D or 3D image data, while the image-taking unit can be an endoscope.

The aligning of the comparative image data and the reference image data can further include aligning the comparative image data and the reference image data by using the patient position data and robot position data of a robot arm coupled with the image-taking unit.

Also, the method can further include, after the aligning of the comparative image data and the reference image data, controlling a display unit to output the reference image data and the comparative image data aligned using the patient position data. Here, the reference image data can be outputted in correspondence with a viewing direction of the image-taking unit.

Also, the aligning of the comparative image data and the reference image data can further include aligning the comparative image data and the reference image data by using a distance from the robot arm, an extending direction, and a viewing direction of the image-taking unit.

Aligning the patient position data and the comparative image data can further include the image-taking unit generating distance information of an object of image-taking by using a plurality of lenses each having a different parallax or can further include the image-taking unit generating distance information of an object of image-taking by using one lens and taking images of the object while moving.

The image processing unit can perform a method of extracting difference image data from the comparative image data, where the difference image data generated in correspondence with a progress of surgery; and reconfiguring the reference image data by subtracting the difference image data from the reference image data.

A surgical navigation apparatus and an operating method thereof according to certain embodiments of the invention can provide in real time images of the lesion taken during surgery, so that these images may be compared with those taken before surgery. The images provided can be outputted in 3D form with respect to the current position of the endoscope and the surrounding structures, to enable more accurate surgery and also provide greater convenience for the surgeon.

Also, when using a surgical navigation apparatus and an operating method thereof according to certain embodiments of the invention, a surgeon performing surgery can view a current image, implemented from the comparative image data, and also view an image taken before surgery, implemented from the reference image data, from the same position and along the same direction. Thus, the surgeon can be informed in real time of how much the surgery has progressed.

Additional aspects, features, and advantages, other than those described above, will be obvious from the claims and written description below.

### BRIEF DESCRIPTION OF THE DRAWINGS

FIG. 1 illustrates a surgical navigation apparatus according to the related art.

FIG. 2 illustrates a surgical navigation apparatus according to an embodiment of the invention.

FIG. 3 is a block diagram of a surgical navigation apparatus according to an embodiment of the invention.

FIG. 4 is a flow diagram of a method of operating a surgical navigation apparatus according to an embodiment of the invention.

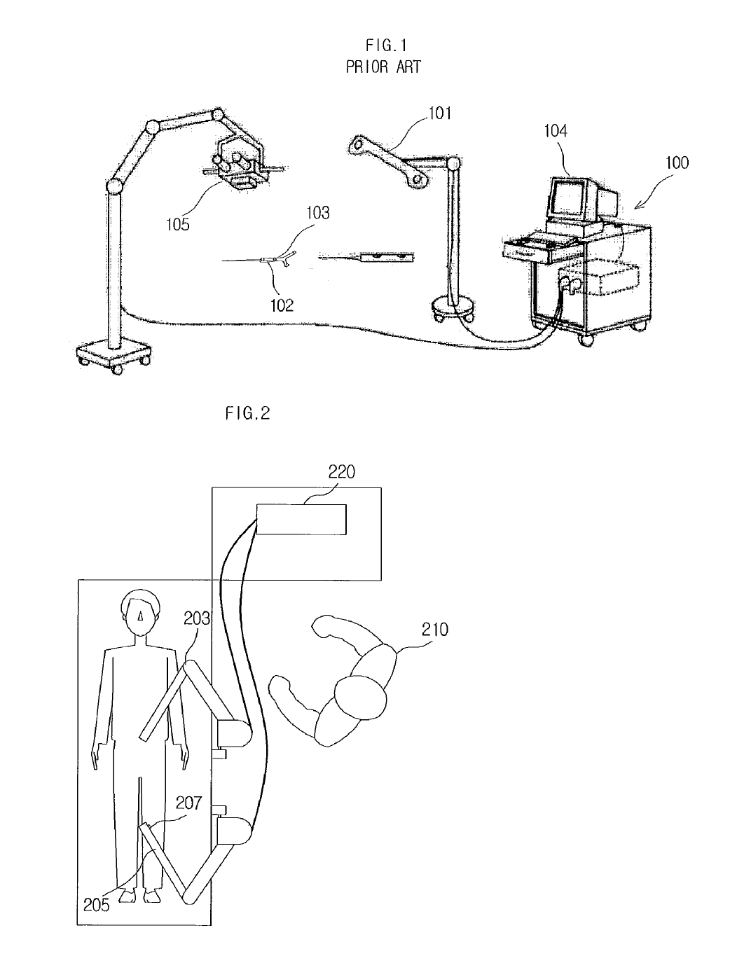

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another.

When a component is said to be “connected to” or “accessing” another component, it is to be appreciated that the two components can be directly connected to or directly accessing each other but can also include one or more other components in-between. The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Also, in providing descriptions referring to the accompanying drawings, those components that are the same or are in correspondence are rendered the same reference numeral regardless of the figure number, and redundant descriptions are omitted. In the written description, certain detailed explanations of related art are omitted, when it is deemed that they may unnecessarily obscure the essence of the present invention.

FIG. 2 illustrates a surgical navigation apparatus according to an embodiment of the invention. Illustrated in FIG. 2 are a robot arm 203, a surgical instrument 205, an image-taking unit 207, a surgeon 210, and a surgical navigation apparatus 220. While the following descriptions will focus on a method of processing images using a surgical robot, the invention is not limited to such robotic surgery, and the invention can be applied, for example, to a surgery-assisting robot equipped with only a camera function.

A feature of this embodiment is an image processing method in which images, i.e. the data of a patient's diagnosis images generated by image-taking before surgery and the image data obtained by an endoscope during surgery, are aligned with each other to provide in real time image information of the lesion for both before surgery and during surgery, so as to enable more accurate surgery and also allow the surgeon to conduct surgery more conveniently.

A patient's diagnosis image generated by image-taking before surgery is an image showing the state, position, etc., of the lesion, and is not particularly limited in type. For example, the diagnosis image can include various images such as CT images, MRI images, PET images, X-ray images, ultrasonic images, etc.

The robot arm 203 may have a surgical instrument 205 and an image-taking unit 207, such as an endoscope, coupled thereto. Here, the endoscope can be a 2D or a 3D endoscope, which can include a rhinoscope, a bronchoscope, an esophagoscope, a gastroscope, a duodenoscope, a rectoscope, a cystoscope, a laparoscope, a thorascope, a mediastinoscope, a cardioscope, etc. The following descriptions will focus on an example in which the image-taking unit 207 is a 3D endoscope,

The surgical navigation apparatus 220 may be an apparatus for providing convenience to a surgeon 210 performing image-guided surgery. The surgical navigation apparatus 220 may output an image, formed by aligning an image taken before surgery and an image taken during surgery, to a display unit.

The surgical navigation apparatus 220 may align the before-surgery image and the during-surgery image by using the patient's reference image data taken before surgery, the patient's position data, and comparative image data of the patient's lesion during surgery. The patient's reference image data may be generated by a certain medical device that takes the diagnosis image described above before surgery with a special marker attached onto the patient. Also, patient position data may be aligned with the reference image data by aligning the positions of marker points attached to the patient's body immediately before surgery with the marker point positions included in the reference image data.

The patient position data can be generated by identifying the position of a certain probe located at the patient's lesion. For example, if the probe is positioned at the lesion or at a particular position on the patient, a certain camera (e.g. an infrared camera) may identify a particular reflector (e.g. an infrared reflector) of the probe and may transmit the position information of the probe to the surgical navigation apparatus 220, whereby the patient position data can be obtained. Of course, the patient position data according to this embodiment can also be generated by methods other than that described above (for example, by way of an optical tracking system (OTS), a magnetic system, an ultrasonic system, etc.).

The method for aligning the reference image data, which is generated beforehand and stored in the surgical navigation apparatus 220, and the patient position data with each other and registering can be implemented in various ways, and the invention is not limited to any particular method. For example, the reference image data and the patient position data can be aligned with each other by mapping the coordinate system of the reference image data, the coordinate system of the camera for generating patient position data, and the coordinate system of the patient position data. This registration procedure can include a procedure of converting points in the patient position data into points in the reference image data.

Afterwards, during surgery, the patient position data described above and comparative image data taken by the image-taking unit 207, which is coupled to the robot arm 203, may be aligned with each other. The comparative image data may be image data generated from a 3D endoscope taking images of the patient's lesion, and can be aligned with the reference image data described above and outputted in real time on a display. Since the image-taking unit 207 is coupled to the robot arm 203, it is possible to identify the position of the robot arm 203 as coordinates, with respect to a marker point attached to the patient. Also, since the distance from one end of the robot aim 203, the extending direction, and the viewing direction of the image-taking unit 207 can be calculated from the initial setting values and modified values, it is also possible to identify the position coordinates and direction of the image-taking unit 207 by using robot position data of the robot arm 203 and the patient position data.

Therefore, since the reference image data may be aligned with the patient position data, and the comparative image data may also be aligned with the patient position data, the comparative image data can consequently be aligned with the reference image data. As such image data can be implemented in 2D or 3D, the reference image data can be outputted that corresponds to the viewing direction of the image-taking unit 207. For example, an image corresponding to the reference image data can be reconfigured according to the viewing direction of the image-taking unit 207 for output. This can be implemented by using the position coordinates and direction information of the image-taking unit 207 calculated for the coordinate system of the reference image data, the coordinate system of the camera for generating patient position data, and the coordinate system of the patient position data, as described above.

Thus, a surgeon performing surgery can view an image taken currently, which is implemented from the comparative image data, and an image taken before surgery, which is implemented from the reference image data, for the same position and in the same direction during surgery, for greater accuracy of the surgery as well as greater convenience.

Also, as the position information of the image-taking unit 207 can be identified relatively by comparing with the position of the information of the robot arm 203, information on the position and viewing direction of one end of the image-taking unit 207 can be identified by using the position data of the robot arm 203. Thus, the surgical navigation apparatus 220 can output the image-taking unit 207 on the screen while outputting the reference image data or the comparative image data. For example, in cases where the image-taking unit 207 is shaped like a rod, the surgical navigation apparatus 220 can additionally display a rod-like shape, corresponding to the image-taking unit 207, in the diagnosis implemented by the reference image data.

Here, the robot arm 203, surgical instrument 205, image-taking unit 207, and surgical navigation apparatus 220 can transmit and receive information by way of wired or wireless communication. Implementing wireless communication can eliminate the hassle caused by wires, to allow greater convenience in performing surgery.

Also, the image-taking unit 207 can generate distance information for an object of the image-taking by using a multiple number of lenses each of which has a different parallax. For example, the image-taking unit 207 can be equipped with two lenses arranged left and right, and by taking an image of an object with different parallaxes, the distance can be identified by using a difference in convergence angle between the left image and right image, and the object of image-taking can be identified in 3D form. The surgical navigation apparatus 220 may receive this 3D information to output the comparative image data. The image outputted from the surgical navigation apparatus 220 may be an image reconfigured from a 2D image or 3D image taken before surgery, and since the reconfigured image received and outputted from the image-taking unit 207 may be of a current 3D form, the surgeon can see in real time how much the surgery has progressed.

Also, according to another embodiment, the image-taking unit 207 can generate distance information for an object of the image-taking by using one lens and taking images while moving. For example, the image-taking unit 207 can identify the object of image-taking in 3D form as described above, by taking images of the object with different parallaxes while moving. As the image-taking unit 207 generates the distance information described above while performing actions of moving forward or backward, rotating, etc., it can identify forms in 3D by using information on the space in which the image-taking unit 207 is positioned.

By using the 3D information implemented from the distance information of the object of image-taking as described above, it is also possible to obtain progress information of the surgery from the diagnosis image. That is, the diagnosis image obtained before surgery and the reconfigured image taken during surgery can be compared and a difference image can be deduced, after which the corresponding difference image can be subtracted from the diagnosis image to output the current progress information of the surgery. For example, if the lesion is a portion where a tumor is formed, and the surgery being conducted is for removing the tumor, then the difference image described above may be an image corresponding to the tumor being removed, and the progress of removing the tumor can be outputted in real time as a reconfigured diagnosis image.

For this purpose, a surgical navigation apparatus 220 according to this embodiment can extract the difference image data generated in correspondence to the surgery progress from the comparative image data taken during surgery, reconfigure the reference image data by subtracting the difference image data from the reference image data, and output the results as the reconfigured diagnosis image. The difference image data can be extracted by comparing the reference image data and comparative image data for the same object of image-taking, or by comparing multiple sets of comparative image data for the same object of image-taking.

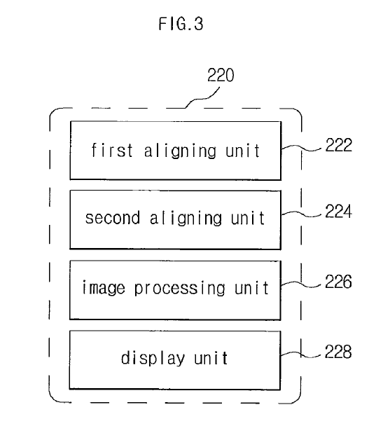

FIG. 3 is a block diagram of a surgical navigation apparatus according to an embodiment of the invention. Illustrated in FIG. 3 is a surgical navigation apparatus 220 that includes a first aligning unit 222, a second aligning unit 224, an image processing unit 226, and a display unit 228.

The first aligning unit 222 may align the patient's position with the reference image data, by using the patient position data and the patent's reference image data generated by image-taking before surgery. As described above, the patient position data and the reference image data may be aligned with each other and registered by the first aligning unit 222. The reference image data and the patient position data can be aligned, for example, by mapping the coordinate system of the reference image data, the coordinate system of the camera for generating the patient position data described above, and the coordinate system of the patient position data.

The second aligning unit 224 may align in real time the patient position data and the comparative image data received from the image-taking unit. That is, the second aligning unit 224 may align the comparative image data, which is taken during surgery by the image-taking unit 207 coupled to the robot aim 203, and the patient position data described above. For example, the second aligning unit 224 can align the patient position data and the comparative image data in real time by calculating the coordinate values of the robot arm 203 and the image-taking unit 207 from the coordinate system of the patient position data. Of course, the coordinate values of the robot arm 203 and the image-taking unit 207 can be calculated by presetting the coordinate system of the robot arm 203 or the coordinate system of the image-taking unit 207 with respect to the coordinate system of the patient position data and then applying the change values. Although the second aligning unit 224 has been denoted differently from the first aligning unit 222 herein, the two can be implemented as the same apparatus. That is, while the first aligning unit 222 and the second aligning unit 224 may be separate components in terms of function, they can be implemented in substantially the same apparatus or with only the specific source code differing.

The image processing unit 226 may align the comparative image data and the reference image data in real time by using the patient position data. The aligned comparative image data and reference image data can be outputted on an adjacent display unit 228 so that the surgeon may easily compare the two.

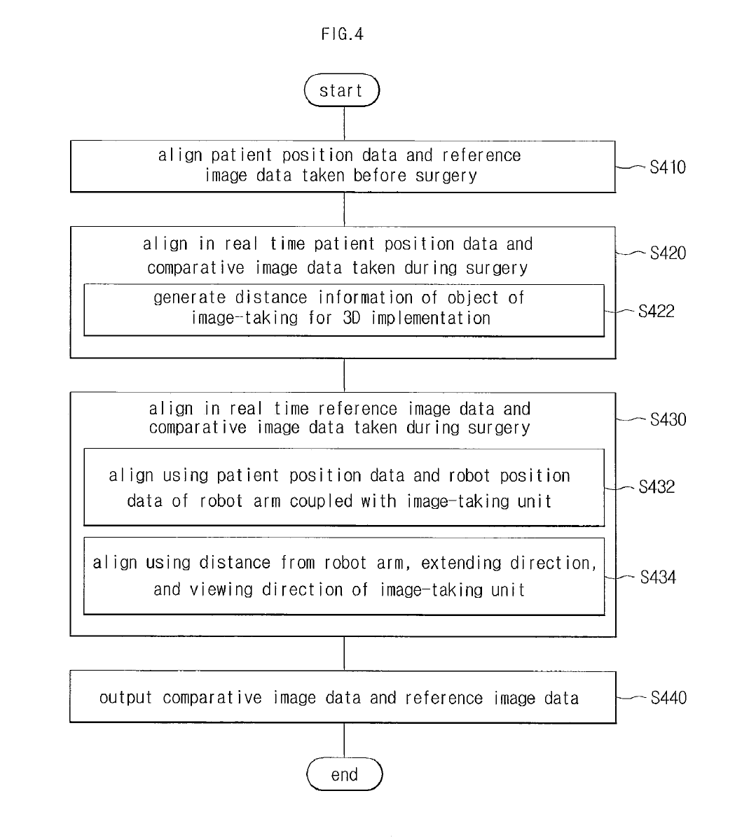

FIG. 4 is a flow diagram of a method of operating a surgical navigation apparatus according to an embodiment of the invention.

In step S410, the first aligning unit 222 may align the patient's position with the reference image data, by using the patient position data and the reference image data generated by image-taking before surgery. As described above, this can be implemented by mapping the coordinate system of the reference image data, the coordinate system of the camera for generating the patient position data, and the coordinate system of the patient position data.

In step S420, the second aligning unit 224 may in real time align the patient position data and the comparative image data received from the image-taking unit 207.

Here, the image-taking unit 207 can generate distance information for the object of image-taking by using multiple lenses having different parallaxes or by taking images while moving, in order to implement a 3D image (step S422). This 3D image can be used in outputting the reference image data in the direction viewed by the image-taking unit 207.

In step S430, the image processing unit 226 may align the comparative image data and the reference image data in real time by using the patient position data. Here, the image processing unit 226 can align the comparative image data and the reference image data by using the robot position data of a robot arm coupled with the image-taking unit 207 and the patient position data (step S432). Also, the image processing unit 226 can align the comparative image data and the reference image data by using the distance from the robot arm 203, the extending direction, and the viewing direction of the image-taking unit 207 (step S434).

In step S440, the surgical navigation apparatus 220 may control the display unit to output the aligned comparative image data and reference image data by using the patient position data, and in this case, the reference image data can be outputted in correspondence with the viewing direction of the image-taking unit.

The description of other details related to the surgical navigation apparatus according to an embodiment of the present invention, including, for example, common platform technology, such as the embedded system, O/S, etc., interface standardization technology, such as the communication protocol, I/O interface, etc., and component standardization technology, such as for actuators, batteries, cameras, sensors, etc., will be omitted, as these are apparent to those of ordinary skill in the art.

The method of operating a surgical navigation apparatus according to an embodiment of the present invention can also be implemented in the form of program instructions executable by various computer means and can be recorded in a computer-readable medium. In other words, the recorded medium can be a medium which can be read by a computer and which includes a program recorded thereon that enables a computer to execute the steps described above.

The computer-readable medium can include program instructions, data files, data structures, etc., alone or in combination thereof. The program instructions recorded on the medium can be those that are specifically designed and configured for the present invention or can be those available to the skilled person in the computer software industry. Examples of the recorded medium readable by a computer include magnetic media such as hard disks, floppy disks, and magnetic tape, optical media such as CD-ROM and DVD's, magneto-optical media such as floptical disks, as well as hardware devices specifically configured to store and perform the program instructions such as ROM, RAM, flash memory, etc.

While the surgical navigation apparatus according to certain embodiments of the invention has been disclosed in the foregoing descriptions for an example that employs a surgical robot and an image-guided surgery system, the invention is not necessarily limited thus. For example, an embodiment of the invention can also be applied to a surgical system using a manual endoscope, and even if one of the components of an image-guided surgery system is implemented differently, such an arrangement can be encompassed by the scope of claims of the present invention if there is no significant difference in overall operation and effect.

For example, certain embodiments of the invention can also be applied to a surgical robot system having a master-slave structure, in which a robot arm, surgical instrument, and image-taking unit coupled to the slave robot is operated by a manipulation of a master interface equipped on the master robot.

While the present invention has been described with reference to particular embodiments, it will be appreciated by those skilled in the art that various changes and modifications can be made without departing from the spirit and scope of the present invention, as defined by the claims appended below.

1. A surgical navigation apparatus comprising:
a first aligning unit configured to align a position of a patient with reference image data by using patient position data and the reference image data corresponding to a diagnosis image of the patient generated by image-taking before surgery;
a second aligning unit configured to align the patient position data and comparative image data corresponding to an endoscope image received from an image-taking unit in real time;
an image processing unit configured to align the comparative image data and the reference image data in real time by using the patient position data; and
a display unit configured to output the reference image data and the comparative image data aligned with the patient position data.

2. A surgical navigation apparatus comprising:
an image processing unit configured to align reference image data corresponding to a diagnosis image of the patient generated by image-taking before surgery and comparative image data corresponding to an endoscope image received from an image-taking unit during surgery in real time; and
a display unit configured to output the reference image data and the comparative image data aligned,
wherein the image processing unit aligns the reference image data and the comparative image data with a coordinate system of a robot arm where the image-taking unit is coupled using a position information of the image-taking unit.

3. The surgical navigation apparatus of claim 1, wherein the image-taking unit generates distance information of an object of image-taking by using a plurality of lenses each having a different parallax.

4. The surgical navigation apparatus of claim 2, wherein the image-taking unit generates distance information of an object of image-taking by using a plurality of lenses each having a different parallax.

5. The surgical navigation apparatus of claim 1, wherein the image processing unit aligns the comparative image data and the reference image data by using the patient position data and robot position data of a robot arm coupled with the image-taking unit.

6. The surgical navigation apparatus of claim 5, wherein the image processing unit aligns the comparative image data and the reference image data by using a distance from the robot arm, an extending direction, and a viewing direction of the image-taking unit.

7. The surgical navigation apparatus of claim 1, wherein the reference image data is outputted in correspondence with a viewing direction of the image-taking unit.

8. The surgical navigation apparatus of claim 2, wherein the reference image data is outputted in correspondence with a viewing direction of the image-taking unit.

9. The surgical navigation apparatus of claim 1, wherein the image-taking unit generates distance information of an object of image-taking by using one lens and taking images of the object while moving.

10. The surgical navigation apparatus of claim 2, wherein the image-taking unit generates distance information of an object of image-taking by using one lens and taking images of the object while moving.

11. The surgical navigation apparatus of claim 1, wherein the image processing unit extracts difference image data from the comparative image data, the difference image data generated in correspondence with a progress of surgery, and wherein the reference image data is reconfigured by subtracting the difference image data from the reference image data.

12. A method of operating a surgical navigation apparatus, by which the surgical navigation apparatus processes an image in real time during surgery, the method comprising:
aligning a position of a patient with reference image data by using patient position data and the reference image data corresponding to a diagnosis image of the patient generated by image-taking before surgery;
aligning the patient position data and comparative image data corresponding to an endoscope image received from an image-taking unit in real time;
aligning the comparative image data and the reference image data in real time by using the patient position data; and
outputting the reference image data and the comparative image data aligned with the patient position data.

13. A method of operating a surgical navigation apparatus, the method comprising:
aligning reference image data corresponding to a diagnosis image of the patient generated by image-taking before surgery and comparative image data corresponding to an endoscope image received from an image-taking unit during surgery in real time; and
outputting the reference image data and the comparative image data aligned,
wherein the reference image data and the comparative image data are aligned with a coordinate system of a robot arm where the image-taking unit is coupled using a position information of the image-taking unit.

14. The method of claim 12, further comprising, after the aligning of the comparative image data and the reference image data:
extracting difference image data from the comparative image data, the difference image data generated in correspondence with a progress of surgery; and
reconfiguring the reference image data by subtracting the difference image data from the reference image data.

15. The method of claim 13, further comprising, after the aligning of the comparative image data and the reference image data:
extracting difference image data from the comparative image data, the difference image data generated in correspondence with a progress of surgery; and
reconfiguring the reference image data by subtracting the difference image data from the reference image data.

16. The method of claim 12, wherein the aligning of the comparative image data and the reference image data further comprises:
aligning the comparative image data and the reference image data by using the patient position data and robot position data of a robot arm coupled with the image-taking unit.

17. The method of claim 16, wherein the aligning of the comparative image data and the reference image data further comprises:
aligning the comparative image data and the reference image data by using a distance from the robot arm, an extending direction, and a viewing direction of the image-taking unit.

18. The method of claim 12, wherein the reference image data is outputted in correspondence with a viewing direction of the image-taking unit.

19. The method of claim 13, wherein the reference image data is outputted in correspondence with a viewing direction of the image-taking unit.

20. The method of claim 12, wherein the aligning of the patient position data and the comparative image data further comprises:
generating distance information, by the image-taking unit, of an object of image-taking by using a plurality of lenses each having a different parallax.

21. The method of claim 12, wherein the aligning of the patient position data and the comparative image data further comprises:
generating distance information, by the image-taking unit, of an object of image-taking by using one lens and taking images of the object while moving.

22. The surgical navigation apparatus of claim 2, wherein the image processing unit extracts difference image data from the comparative image data, the difference image data generated in correspondence with a progress of surgery, and wherein the reference image data is reconfigured by subtracting the difference image data from the reference image data.
